(19) United States
(12) Patent Application Publication
(10) Pub. No.: US 2021/0380518 A1
(43) Pub. Date: Dec. 9, 2021

(54) COMPOUNDS AND METHODS OF USING COMPOUNDS FOR TREATMENT OF RESPIRATORY DISEASES

(71) Applicant: Kambiz Shekdar, Brooklyn, NY (US)

(72) Inventor: Kambiz Shekdar, Brooklyn, NY (US)

(73) Assignee: Kambiz Shekdar, Brooklyn, NY (US)

(21) Appl. No.: 17/342,148

(22) Filed: Jun. 8, 2021

Related U.S. Application Data
(60) Provisional application No. 63/036,165, filed on Jun. 8, 2020.

(51) Int. Cl. C07C 50/34; A61K 45/06; A61P 31/14

(52) U.S. Cl. CPC C07C 50/34; A61P 31/14; A61K 45/06

(57) ABSTRACT

Provided herein are compositions and methods of using compositions for treating respiratory diseases in a subject in need of such treatment. In particular, provided herein are compositions and methods of using compositions for the treatment of SARS-CoV-2 (Covid-19). More particularly, provided herein are compositions comprising ENaC agonists for treating SARS-CoV-2 (Covid-19).

### FIELD

Provided herein are compositions and methods of using compositions for treating respiratory diseases in a subject in need of such treatment. In particular, provided herein are compositions and methods of using compositions for the treatment of SARS-CoV-2 (Covid-19). More particularly, provided herein are compositions comprising ENaC agonists for treating SARS-CoV-2 (Covid-19).

### BACKGROUND

Pulmonary edema (PE) is a feature of some respiratory diseases, including Covid-19 and is characterized by filling of the air sacs in the lung with fluid. Epithelial Na⁺ channel (ENaC is expressed on the apical surface of epithelial cells in lung alveoli where it plays a key role in regulating fluid levels. While clinical trials using indirect mechanisms to activate ENaC have been pursued to address PE, no potentiators of ENaC were previously known for therapeutic use [M. Fornius, “Treatment of Pulmonary Edema by ENaC Activators/Stimulators,” Current Molecular Pharmacology, vol. 6, pp. 13-27, 2013.]

Chromovert® Technology enabled the creation of stable αβγ-ENaC cell-based assays [K. Shekdar and J. Langer, “Cell lines expressing ENaC and methods of using them”. U.S. Pat. No. 9,534,035].

High-throughput screening of numerous compounds, including pharmaceutically active compounds and food and flavor ingredients capable of modulating, in particular, enhancing ENaC activity were identified using ENaC cell-based assays.

The need exists to evaluate these ENaC enhancers for their ability to prevent and treat respiratory disease and in particular Covid-19.

### SUMMARY

In one embodiment, provided herein is a composition comprising a compound selected from: a compound of Formula (IV)

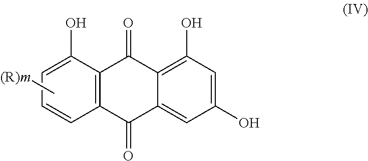

or a comestibly or biologically acceptable salt, or an enantiomer, diastereomer, or solvate thereof, or a combination of any of the foregoing compounds; wherein as valence and stability permit:

R is each independently hydrogen, halogen, C1-C6 alkyl, Cl-C6 alkoxy, or hydroxyl;

m is 1, 2, or 3; or

a combination of any one of compounds (a)-(e);

and at least one pharmaceutically acceptable carrier or diluent.

In one embodiment, provided herein is a composition wherein the compound of Formula (IV) is 1,3,8-trihydroxy-6-methylanthraquinone (Compound 1), having the following structure:

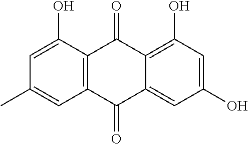 [SMILES: Cc1cc(O)c2c(c1)C(=O)c1cc(O)cc(O)c1C2=O]

In one embodiment, a composition wherein the compound is Emodin.

In one embodiment the compositions disclosed herein are in an aqueous solution.

In one embodiment, the compositions disclosed herein further comprise one or more other active agents selected from the group of antibiotics, vaccines, decongestants (nasal or bronchial), rhDNase, non-steroidal antiinflammatory agents (NSAIDs), steroids, antiviral agents, elastase inhibitors, gene therapy agents, chloride channel activators and bronchodilators.

In one embodiment, the compositions disclosed herein are for use in a method of preventing or treating respiratory disease.

In one embodiment, the respiratory disease is characterized by one or more of the symptoms selected from fluid dysregulation, pulmonary edema, lack of fluid clearance, excessive fluid build-up, poor mucociliary clearance, mucostasis, breathing difficulties, cough, chest pain, and fatigue.

In one embodiment, the respiratory disease is selected from cystic fibrosis, SARS (severe acute respiratory syndrome), MERS (middle east respiratory syndrome), ARDS (acute respiratory distress syndrome), pneumonia, infection, pulmonary infection, viral infection, SARS-CoV-2 (Covid-19), bronchiectasis, chronic bronchitis, and chronic obstructive pulmonary disease (COPD), high altitude pulmonary edema (HAPE).

In one embodiment, the respiratory disease is SARS-CoV-2 (Covid-19).

In one embodiment, the composition disclosed herein is formulated for administration to the respiratory system by the pulmonary route.

In one embodiment, the composition disclosed herein is formulated for administration by any one of endotracheal intubation, intratracheal installation, intratracheal delivery of lipososmes, insufflation, nebulization, dry powder inhalation and aerosol inhalation.

In one embodiment, the composition disclosed herein is formulated for administration by diffusion in the air.

In one embodiment, the compositions disclosed herein is formulated for administration by infusion into cloth selected from tissues, handkerchiefs, napkins, face masks, mufflers, scarfs, wraps, bandanas, ties, shawls, hijabs, and veils.

In one embodiment, the composition disclosed herein is formulated for administration by injection, oral or rectal route.

In one embodiment, the composition disclosed herein is administered by inhalation.

In one embodiment, the composition disclosed herein is formulated for dry powder inhalation or aerosol inhalation, together with a propellant selected from hydrofluroalkanes, chlorofluorocarbons, propane and nitrogen, or mixtures thereof.

In one embodiment, an inhalation device is loaded with the pharmaceutical composition of any one of the compositions disclosed herein.

In one embodiment, the inhalation device is a dry powder inhaler, metered dose inhaler, jet nebulizer or ultrasonic nebulizer.

In one embodiment, a method of treating a respiratory disease, the method comprises administering to a subject in need of such treatment a therapeutically effective amount of a compound selected from: a compound of Formula (IV), and Compound 1; or a comestibly or biologically acceptable salt, or an enantiomer or diastereomer thereof, or a combination of any of the foregoing compounds.

In one embodiment, a method of treating a respiratory disease, the method comprises administering to a subject in need of such treatment a therapeutically effective amount of Emodin, or a pharmaceutically acceptable salt, or a stereoisomeric or tautomeric form thereof.

In one embodiment, a method of treating a respiratory disease, the method comprises administering to a subject in need of such treatment a therapeutically effective amount of a compound wherein the compound is an ENaC agonist.

In one embodiment, the respiratory disease is characterized by one or more of the symptoms selected form poor mucociliary clearance, mucostasis, breathing difficulties, cough, chest pain, and fatigue.

In one embodiment, a method as disclosed herein wherein the respiratory disease is selected from SARS-CoV-2 (Covid-19), cystic fibrosis, bronchiecstasis, chronic bronchitis, and chronic obstructive pulmonary disease (COPD).

In one embodiment, a method as disclosed herein wherein the respiratory disease is SARS-CoV-2 (Covid-19).

In one embodiment, a method as disclosed herein wherein the administration is to the respiratory system by the pulmonary route.

In one embodiment, a method as disclosed herein wherein the administration is by one or more treatments selected from endotracheal intubation, intratracheal installation, intratracheal deliver of liposomes, insufflation, nebulization, dry powder inhalation and aerosol inhalation.

In one embodiment, a method as disclosed herein wherein the administration is by dry powder inhalation or aerosol inhalation, together with a propellant selected rom hydrofluroalkanes, chlorofluorocarbons, propane, nitrogen, or a mixture thereof.

In one embodiment, an ENaC agonist is for use in a method of treating a respiratory disease.

In one embodiment, the ENaC agonist as disclosed herein is selected from modulators, including allosteric modulators, potentiators, enhancers, activators, and blockers (wherein a blocker of one form of ENaC is an allosteric modulator, potentiator, enhancer, or activator of another form of ENaC) or any combination of the foregoing.

In one embodiment, the ENaC agonist as disclosed herein is an ENaC enhancer.

In one embodiment, the agonist as disclosed herein is selected from a compound of Formula (IV), and Compound 1; or a comestibly or biologically acceptable salt, or an enantiomer or diastereomer thereof, or a combination of any of the foregoing compounds.

In one embodiment, the ENaC agonist as disclosed is Emodin.

In one embodiment, the ENaC agonist as disclosed herein further comprises one or more pharmaceutically acceptable carriers or diluents.

In one embodiment, the ENaC agonist as disclosed herein is in an aqueous solution.

In one embodiment, the ENaC agonist as disclosed herein also comprises one or more other active agents selected from antibiotics, vaccines, bronchial decongestants, rhDNase, non-steroidal anti-inflammatory agents (NSAIDs), steroids, antiviral agents, elastase inhibitors, gene therapy agents, chloride channel activators and bronchodilators.

In one embodiment, the use of a compound selected from:

a compound of Formula (IV), and Compound 1; or a comestibly or biologically acceptable salt, or an enantiomer or diastereomer thereof, or a combination of any of the foregoing compounds is for the manufacture of a medicament for the treatment of respiratory disease.

In one embodiment, the use of a compound wherein the compound is Emodin.

In one embodiment, an article of manufacture comprises packaging material and a pharmaceutical agent contained within said packaging material, wherein said packaging material comprises a label which indicates said pharmaceutical may be administered, for a sufficient term at an effective dose, for treating respiratory conditions together with a pharmaceutically acceptable carrier, wherein the pharmaceutical agent comprises one or more compounds as disclosed herein or a pharmaceutically acceptable salt, or a tautomeric form thereof.

### DETAILED DESCRIPTION

A “Composition” or “Compositions” as used herein comprise a compound that is known to enhance ENaC, a compound selected from a compound of Formula (IV), and Compound 1; or a comestibly or biologically acceptable salt, or an enantiomer or diastereomer thereof, or a combination of any of the foregoing compounds; and at least one pharmaceutically acceptable carrier or diluent.

A “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the Compounds include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Others are well known in the art, see for example, Remington's Pharmaceutical Sciences, 18th eds., Mack Publishing, Easton Pa. (1990) or Remington: The Science and Practice of Pharmacy, 19th eds., Mack Publishing, Easton Pa. (1995).

A “stereoisomer” or “stereoisomeric form” refers to one stereoisomer of a Compound that is substantially free of other stereoisomers of that Compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The Compounds can have chiral centers and can occur as racemates, individual enantiomers or diastereomers, and mixtures thereof. All such isomeric forms are included within the embodiments disclosed herein, including mixtures thereof. The use of stereomerically pure forms of such Compounds, as well as the use of mixtures of those forms, are encompassed by the embodiments disclosed herein. For example, mixtures comprising equal or unequal amounts of the enantiomers of a particular Compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

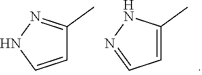

As readily understood by one skilled in the art, a wide variety of functional groups and other structures may exhibit tautomerism and all tautomers of the Compounds provided herein are within the scope of the present disclosure.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include, but are not limited to, phenyl, naphthyl and the like.

An “alkyl” group is a saturated straight chain or branched non-cyclic hydrocarbon having, for example, from 1 to 12 carbon atoms, 1 to 9 carbon atoms, 1 to 6 carbon atoms, 1 to 4 carbon atoms, or 2 to 6 carbon atoms. Representative alkyl groups include -methyl, -ethyl, -n-propyl, -n-butyl, -n-pentyl and -n-hexyl; while branched alkyls include -isopropyl, -sec-butyl, -iso-butyl, -tert-butyl, -iso-pentyl, 2-methylpentyl, 3-methylpentyl, 4-methylpentyl, 2,3-dimethylbutyl and the like.

An “alkenyl” group is a partially unsaturated straight chain or branched non-cyclic hydrocarbon having, for example, from 3 to 6 carbon atoms, 3 to 4 carbon atoms, or 3 carbon atoms. Representative alkenyl groups include allyl, propenyl and the like. An “alkynyl” group is a partially unsaturated straight chain or branched non-cyclic hydrocarbon having, for example, from 3 to 6 carbon atoms, 4 to 6 carbon atoms, or 3 carbon atoms. Representative alkynyl groups include propynyl, butynyl and the like. A “cycloalkyl” group is a saturated cyclic alkyl group of from 3 to 12 carbon atoms having a single cyclic ring or multiple condensed or bridged rings. In some embodiments, the cycloalkyl group has 4 to 12 ring members, whereas in other embodiments the number of ring carbon atoms ranges, for example, from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, and the like, or multiple or bridged ring structures such as adamantyl and the like.

It should also be noted the Compounds provided herein can contain unnatural proportions of atomic isotopes at one or more of the atoms. For example, the Compounds may be radiolabeled with radioactive isotopes, such as for example tritium (3H), iodine-125 (1²⁵I), sulfur-35 (3⁵ S), or carbon-14 (1⁴ C), or may be isotopically enriched, such as with deuterium (2H), carbon-13 (¹³C), or nitrogen-15 (1⁵N). As used herein, an “isotopologue” is an isotopically enriched Compound. The term “isotopically enriched” refers to an atom having an isotopic composition other than the natural isotopic composition of that atom. “Isotopically enriched may also refer to a Compound containing at least one atom having an isotopic composition other than the natural isotopic composition of that atom. The term “isotopic composition” refers to the amount of each isotope present for a given atom. Radiolabeled and isotopically enriched Compounds are useful as therapeutic agents, e.g., cancer and inflammation therapeutic agents; research reagents, e.g., binding assay reagents; and diagnostic agents, e.g., in vivo imaging agents. All isotopic variations of the Compounds as described herein, whether radioactive or not, are intended to be encompassed within the scope of the embodiments provided herein. In some embodiments, there are provided isotopologues of the Compounds, for example, the isotopologues are deuterium, carbon-13, or nitrogen-15 enriched Compounds.

A “subject in need thereof” refers to a mammal (e.g., human, dog, horse, or cat) in need of treatment with any method provided herein. In one embodiment the subject is a patient. The term “agonist” refers to modulators, including allosteric modulators, potentiators, enhancers, activators, and blockers, or any combination of the foregoing

In one embodiment, provided herein is a composition comprising a compound selected from:

a compound of Formula (IV)

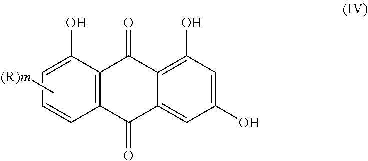

or a comestibly or biologically acceptable salt, or an enantiomer, diastereomer, or solvate thereof, or a combination of any of the foregoing compounds; wherein as valence and stability permit:

R is each independently hydrogen, halogen, C1-C6 alkyl, C1-C6 alkoxy, or hydroxyl;

m is 1, 2, or 3; or

a combination of any one of compounds of the foregoing compounds;

and at least one pharmaceutically acceptable carrier or diluent.

In one embodiment, provided herein is a composition wherein the compound of Formula (IV) is a compound wherein R of the compound of Formula (IV) is —CH₃ and m is 1.

In one embodiment, provided herein is a composition wherein the compound of Formula (IV) is 1,3,8-trihydroxy-6-methylanthraquinone (Compound 1), having the following structure:

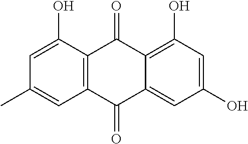 [SMILES: Cc1cc(O)c2c(c1)C(=O)c1cc(O)cc(O)c1C2=O]

In one embodiment, provided here is a composition wherein the compound is Emodin. In one embodiment the compositions disclosed herein are in an aqueous solution.

In one embodiment, the compositions disclosed herein the composition comprises one or more other active agents selected from the group of antibiotics, vaccines, decongestants (nasal or bronchial), rhDNase, non-steroidal antiinflammatory agents (NSAIDs), steroids, antiviral agents, elastase inhibitors, gene therapy agents, chloride channel activators and bronchodilators.

In one embodiment, the compositions disclosed herein are for use in a method of preventing or treating respiratory disease.

In one embodiment, the respiratory disease is characterized by one or more of the symptoms selected from fluid dysregulation, pulmonary edema, lack of fluid clearance, excessive fluid build-up, poor mucociliary clearance, mucostasis, breathing difficulties, cough, chest pain, and fatigue.

In one embodiment, the respiratory disease is selected from cystic fibrosis, SARS (severe acute respiratory syndrome), MERS (middle east respiratory syndrome), ARDS (acute respiratory distress syndrome), pneumonia, infection, pulmonary infection, viral infection, SARS-CoV-2 (Covid-19), bronchiectasis, chronic bronchitis, and chronic obstructive pulmonary disease (COPD), high altitude pulmonary edema (HAPE).

In one embodiment, the respiratory disease is SARS-CoV-2 (Covid-19).

In one embodiment, the composition disclosed herein is formulated for administration to the respiratory system by the pulmonary route.

In one embodiment, the composition disclosed herein is formulated for administration by any one of endotracheal intubation, intratracheal installation, intratracheal delivery of liposomes, insufflation, nebulization, dry powder inhalation and aerosol inhalation, and inhalation of diffuse compounds where the compounds are volatile.

In one embodiment, the composition disclosed herein is formulated for administration by injection, oral or rectal route.

In one embodiment, the composition disclosed herein is administered by inhalation.

In one embodiment, the composition disclosed herein is formulated for dry powder inhalation or aerosol inhalation, together with a propellant selected from hydrofluroalkanes, chlorofluorocarbons, propane and nitrogen, or mixtures thereof.

In one embodiment, an inhalation device loaded with the pharmaceutical composition of any one of the compositions disclosed herein.

In one embodiment, the inhalation device is a dry powder inhaler, metered dose inhaler, jet nebulizer or ultrasonic nebulizer.

In one embodiment, a method of treating a respiratory disease, the method comprising administering to a subject in need of such treatment a therapeutically effective amount of a compound selected from: Emodin, a compound of Formula (IV), and Compound 1; or a comestibly or biologically acceptable salt, or an enantiomer or diastereomer thereof, or a combination of any of the foregoing compounds.

In one embodiment, a method of treating a respiratory disease, the method comprises administering to a subject in need of such treatment a therapeutically effective amount of Emodin, or a pharmaceutically acceptable salt, or a stereoisomeric or tautomeric form thereof.

In one embodiment, a method of treating a respiratory disease, the method comprises administering to a subject in need of such treatment a therapeutically effective amount of a compound wherein the compound is an ENaC agonist.

In one embodiment, the respiratory disease is characterized by one or more of the symptoms selected form poor mucociliary clearance, mucostasis, breathing difficulties, cough, chest pain, and fatigue.

In one embodiment, a method as disclosed herein wherein the respiratory disease is selected from SARS-CoV-2 (Covid-19), cystic fibrosis, bronchiecstasis, chronic bronchitis, and chronic obstructive pulmonary disease (COPD).

In one embodiment, a method as disclosed herein wherein the respiratory disease is SARS-CoV-2 (Covid-19).

In one embodiment, a method as disclosed herein wherein the administration is to the respiratory system by the pulmonary route.

In one embodiment, a method as disclosed herein wherein the administration is by one or more treatments selected from endotracheal intubation, intratracheal installation, intratracheal deliver of lipososmes, insufflation, nebulization, dry powder inhalation and aerosol inhalation.

In one embodiment, a method as disclosed herein wherein the administration is by dry powder inhalation or aerosol inhalation, together with a propellant selected rom hydrofluroalkanes, chlorofluorocarbons, propane, nitrogen, or a mixture thereof.

In one embodiment, an ENaC agonist is for use in a method of treating a respiratory disease.

In one embodiment, the ENaC agonist as disclosed herein is selected from modulators, potentiators, enhancers, activators, and blockers, or any combination of the foregoing.

In one embodiment, the ENaC agonist as disclosed herein is an ENaC enhancer.

In one embodiment, the agonist as disclosed herein is selected from a compound Emodin, a compound of Formula (IV), and Compound 1; or a comestibly or biologically acceptable salt, or an enantiomer or diastereomer thereof, or a combination of any of the foregoing compounds.

In one embodiment, the ENaC agonist as disclosed is at least one compound is Emodin.

In one embodiment, the ENaC agonist as disclosed herein further comprises one or more pharmaceutically acceptable carriers or diluents.

In one embodiment, the ENaC agonist as disclosed herein is in an aqueous solution.

In one embodiment, the ENaC agonist as disclosed herein also comprises one or more other active agents selected from antibiotics, vaccines, bronchial decongestants, rhDNase, non-steroidal anti-inflammatory agents (NSAIDs), steroids, antiviral agents, elastase inhibitors, gene therapy agents, chloride channel activators and bronchodilators.

In one embodiment, as disclosed herein the therapeutically effective amount is effective to alleviate at least one symptom associated with said respiratory disease in a subject, wherein a compound according to the present disclosure, or a pharmaceutically acceptable salt, or a tautomeric form thereof, shows a reduction in at least one symptom associated with said inflammatory condition at a dose between 0.01 ng/kg and 10,000 ng/kg, at a dose between 0.1 ng/kg and 1,000 ng/kg, at a dose between 0.5 ng/kg and 100 ng/kg, at a dose between 1 ng/kg to 50 ng/kg, at a dose between 0.01 μg/kg and 10,000 μg/kg, at a dose between 0.1 μg/kg and 1,000 μg/kg, at a dose between 0.5 μg/kg and 100 μg/kg, at a dose between 1 μg/kg to 50 μg/kg, at a dose between 0.01 mg/kg and 10,000 mg/kg, at a dose between 0.1 mg/kg and 1,000 mg/kg, at a dose between 0.5 mg/kg and 100 mg/kg, or at a dose between 1 mg/kg to 50 mg/kg.

In one embodiment, the use of a compound selected from:

a compound of Formula (IV), and Compound 1; or a comestibly or biologically acceptable salt, or an enantiomer or diastereomer thereof, or a combination of any of the foregoing compounds for the manufacture of a medicament for the treatment of respiratory disease.

In one embodiment, the use of a compound wherein the compound is Emodin.

In one embodiment, an article of manufacture comprising packaging material and a pharmaceutical agent contained within said packaging material, wherein said packaging material comprises a label which indicates said pharmaceutical may be administered, for a sufficient term at an effective dose, for treating respiratory conditions together with a pharmaceutically acceptable carrier, wherein the pharmaceutical agent comprises one or more compounds as disclosed herein or a pharmaceutically acceptable salt, or a tautomeric form thereof.

In a certain embodiment, the compositions disclosed herein are those wherein the composition comprises Emodin (Compound 1) [1,3,8-trihydroxy-6-methylanthraquinone (cas no. 518-82-1)] or a pharmaceutically acceptable salt, or a stereoisomeric or tautomeric form thereof. Emodin is available as E7881 from Frangula bark, ≥90% (HPLC) Sigma-Aldrich].

In one embodiment, the compounds provided herein are agonists of ENaC.

Any assay known to the skilled artisan can be used to test the effect of a compound provided herein on ENaC (K. Shekdar and J. Langer, “Cell lines expressing ENaC and methods of using them”. U.S. Pat. No. 9,534,035.

### Respiratory Diseases

The intended respiratory diseases include diseases of the lung, and airways (bronchioles, bronchi), but are not limited to those diseases that are characterised by poor mucociliary clearance and/or mucostasis.

In one embodiment, the disease is selected from the group comprising COVID-19, pulmonary edema, cystic fibrosis, bronchiectasis, chronic bronchitis, chronic obstructive pulmonary disease (COPD).

In one embodiment, the respiratory disease is COVID-19.

### Additional Therapeutic Agents, Carrier and Diluents

In one embodiment, the (ENaC) agonist further comprises one or more pharmaceutically acceptable carriers or diluents. Herein, and throughout, the term “and/or” indicates that either one or both of the active ingredients may include the additional feature. In this case specifically, either one or both of the active ingredients may further comprise one or more pharmaceutically acceptable carriers or diluents. Where the (ENaC) agonist and the hyper-osmotic agent or purinergic agonist are present in a single composition this term indicates that the composition includes the additional feature. In this case specifically a composition comprising the ENaC agonist and the hyperosmotic agent or purinergic agonist may comprise one or more pharmaceutically acceptable carriers or diluents.

In another embodiment, the (ENaC) agonist may be in aqueous solution.

In a further embodiment the (ENaC) agonist and/or the hyperosmotic agent or purinergic agonist also comprises one or more other active agents selected from the group of antibiotics, vaccines, decongestants (nasal or bronchial), rhDNase, non-steroidal anti-inflammatory agents (NSAIDs), steroids, antiviral agents, elastase inhibitors, gene therapy agents, chloride channel activators and bronchodilators.

Combined, separate or sequential administration throughout all uses and methods of the disclosure, the (ENaC) agonist and the hyperosmotic agent or purinergic agonist may be for combined, separate or sequential administration.

Combined administration indicates that the (ENaC) agonist and the hyperosmotic agent or purinergic agonist are administered together as a single composition.

Separate administration indicates that ENaC agonist and the hyperosmotic agent or purinergic agonist are administered individually. In this embodiment ENaC agonist and the hyperosmotic agent or purinergic agonist may be administered at the same time or at different times, but are administered as individual compositions.

Sequential administration indicated that the ENaC agonist and the hyperosmotic agent or purinergic agonist are administered separately, in either order, with a temporal separation between administration of the two active agents. Herein the ENaC agonist may be administered before or after the hyperosmotic agent or purinergic agonist and the hyperosmotic agent or purinergic agonist may be administered before or after the ENaC agonist. In certain embodiments administration of the two active agents may be separated by 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 40 minutes, 50 minutes, 60 minutes, 90 minutes, 2 hours, or 3 hours.

### Methods of Administration

In one embodiment, the ENaC agonist and/or the hyperosmotic agent or purinergic agonist may be formulated for administration to the respiratory system by the pulmonary route. In one embodiment, the ENaC agonist and/or the hyperosmotic agent or purinergic agonist is formulated for administration by a method including but not limited to intratracheal installation (delivery of solution into the airways by syringe), intratracheal delivery ofliposomes, insufflation (administration of powder formulation by syringe or any other similar device into the airways), nebulization, dry powder inhalation and aerosol inhalation. Aerosols (e.g. jet or ultrasonic nebulizers, metered-dose inhalers (MDis), and dry-powder inhalers (DPis) can also be used in intranasal applications as well as for pulmonary administration.

The compounds disclosed herein can be used in the form of a pharmaceutical preparation, for example, in solid, semi-solid or liquid form, that contains at least one of the compounds disclosed herein as a bioactive component, alone or in combination with an anti-inflammatory compound; in admixture with a carrier, vehicle or an excipient suitable for enteral or parental administration. Such anti-inflammatory compounds useful in this regard include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; and other anti-inflammatory agents including, but not limited to, colchicine, allopurinol, probenecid, sulfinpyrazone and benzbromarone.

In addition, the compounds disclosed herein may be compounded, for example with a pharmaceutically acceptable carrier or vehicle for solid compositions such as tablets, pellets or capsules; capsules containing liquids; suppositories; solutions; emulsions; aerosols; sprays; suspensions or any other form suitable for use. Suitable carriers and vehicles include, for example, sterile water, sterile physiological saline, gum acacia, gelatin, starch paste, talc, keratin, colloidal silica, urea and the like. In addition, auxiliary, stabilizing, thickening, lubricating and coloring agents may be used. The compounds disclosed herein are present in the compositions in a therapeutically effective amount, i.e., an amount sufficient to restore normal mucosal secretions.

The compositions of this invention may be administered by a variety of methods including orally, sublingually, intranasally, intramuscularly, intravenously, subcutaneously, intravaginally, transdermally, rectally, by inhalation, by infusion into cloth, or as a mouthwash in dosage unit formulations containing conventional nontoxic pharmaceutically acceptable carriers, adjuvants, and vehicles as desired. Topical administration may also involve the use of transdermal administration such as transdermal patches or ionophoresis devices. The preferred mode of administration is left to the discretion of the practitioner, and will depend in-part upon the desired site of action.

In some embodiments the compositions disclosed herein may be administered by infusion into cloth selected from tissues, handkerchiefs, napkins, face masks, mufflers, scarfs, wraps, bandanas, ties, shawls, hijabs, and veils.

For example, when Covid-19, chronic bronchitis or asthma affects the function of the lungs, the compounds disclosed herein can be administered as an atomized aerosol, via a nebulizer, or via perfusion in a fluorocarbon or synthetic pulmonary surfactant; alternatively, the compounds or compositions disclosed herein can be administered intravenously directly. Thus, the active compounds disclosed herein may be administered to the lungs of a patient by any suitable means, but are preferably administered by generating an aerosol comprised of respirable particles, the respirable particles comprised of the active compound, which particles the subject inhales. The respirable particles may be liquid or solid. The particles may optionally contain other therapeutic ingredients such as a sodium channel blocker as noted above, with the sodium channel blocker included in an amount effective to inhibit the reabsorption of water from airway mucous secretions. The particles may optionally contain other therapeutic ingredients such as antibiotics as described in U.S. Pat. Nos. 5,512,269 and 5,716,931 or Uridine Triphosphate Analogs as described in U.S. Pat. No. 5,292,498, nitric oxide inhibitors as described in U.S. Pat. No. 5,859,058, dinucleotides as described in U.S. Pat. No. 5,935,555, or organic acids as described in U.S. Pat. No. 5,908,611. Particles comprised of active compound for practicing the present invention should include particles of respirable size: that is, particles of a size sufficiently small to pass through the mouth and larynx upon inhalation and into the bronchi and alveoli of the lungs. In general, particles ranging from about 0.5 to 10 microns in size (more particularly, less than about 5 microns in size) are respirable. Particles of non-respirable size which are included in the aerosol tend to deposit in the throat and be swallowed, and the quantity of non-respirable particles in the aerosol is preferably minimized. For nasal administration, a particle size in the range of 10-500 microns is preferred to ensure retention in the nasal cavity.

Liquid pharmaceutical compositions of active compound for producing an aerosol can be prepared by combining the active compound with a suitable vehicle, such as sterile pyrogen free water. Other therapeutic compounds, such as a sodium channel blocker, may optionally be included. Solid particulate compositions containing respirable dry particles of micronized active compound may be prepared by grinding dry active compound with a mortar and pestle, and then passing the micronized composition through a 400 mesh screen to break up or separate out large agglomerates. A solid particulate composition comprised of the active compound may optionally contain a dispersant that serves to facilitate the formation of an aerosol. A suitable dispersant is lactose, which may be blended with the active compound in any suitable ratio (e.g., a 1 to 1 ratio by weight). Again, other therapeutic compounds may also be included.

The dosage of active compound for prophylaxis or treatment of lung disease will vary depending on the condition being treated and the state of the subject, but generally may be an amount sufficient to achieve dissolved concentrations of active compound on the airway surfaces of the subject of from about 10⁻¹² to 10⁻¹ mole/liter, from about 10⁻⁹ to 10⁻³ moles/liter, from about 10⁻⁸ to about 10⁻⁴ moles/liter, or from 10⁻⁷ to 10⁻⁵ moles/liter. Depending on the solubility of the particular formulation of active compound administered, the daily dose may be divided among one or several unit dose administrations. Preferably, the daily dose is a single unit dose, which is preferably administered from 1 to 5 times a day to 1 to 3 times a week. Treatments may continue week to week on a chronic basis as necessary (i.e., the active agent can be administered chronically). Administration of the active compounds may be carried out therapeutically (i.e., as a rescue treatment) or prophylactically. If the compounds are administered prophylactically, they may be administered before substantial lung blockage due to retained mucus secretions has occurred, or at a time when such retained secretions have been at least in part removed.

Aerosols of liquid particles comprising the active compound(s) as disclosed herein may be produced by any suitable means, such as with a nebulizer. See, e.g., U.S. Pat. No. 4,501,729. Nebulizers are commercially available devices that transform solutions or suspensions of the active ingredient into a therapeutic aerosol mist either by means of acceleration of a compressed gas, typically air or oxygen, through a narrow venturi orifice or by means of ultrasonic agitation. Suitable formulations for use in nebulizers consist of the active ingredient in a liquid carrier, the active ingredient comprising up to 40% w/w of the formulation, but preferably less than 20% w/w. the carrier is typically water or a dilute aqueous alcoholic solution, preferably made isotonic with body fluids by the addition of, for example, sodium chloride. Optional additives include preservatives if the formulation is not prepared sterile, for example, methyl hydroxybenzoate, antioxidants, flavoring agents, volatile oils, buffering agents and surfactants. Aerosols of solid particles comprising the active compound may likewise be produced with any solid particulate medicament aerosol generator. Aerosol generators for administering solid particulate medicaments to a subject produce particles that are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation. A second type of illustrative aerosol generator comprises a metered dose inhaler. Metered dose inhalers are pressurized aerosol dispensers, typically containing a suspension or solution formulation of the active ingredient in a liquefied propellant. During use these devices discharge the formulation through a valve adapted to deliver a metered volume, typically from 10 to 150 ul, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents. The aerosol, whether formed from solid or liquid particles, may be produced by the aerosol generator at a rate of from about 10 to 150 liters per minute, more preferably from about 30 to 150 liters per minute, and most preferably about 60 liters per minute. Aerosols containing greater amounts of medicament may be administered more rapidly.

Compositions for oral delivery may be in the form of tablets, pills, troches, lozenges, aqueous or oily suspensions, granules or powders, emulsions, capsules, syrups or elixirs. Orally administered compositions may contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, compositions in tablet form may be coated to delay disintegration and absorption in the gastrointestinal tract thereby providing a sustained action over an extended period of time. Selectively permeable membranes surrounding an osmotically active driving com-pound are also suitable for orally administered compositions. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Injectable preparations, for example, sterile injectable aqueous or oleagenous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols, which are solid at ordinary temperatures but liquid at the rectal temperature and will therefore melt in the rectum and release the drug.

Aqueous suspensions containing the compound(s) disclosed herein may also contain one or more preservatives, such as, for example, ethyl or n-propyl-p-hydroxy-benzoate, one or more coloring agents, flavoring agents or sweetening agents.

The compounds of the present disclosure can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition a compound of the present disclosure, stabilizers, preservatives, excipients, and the like. Useful lipids are for example phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W. (1976), p. 33 et seq.

In a further embodiment, the present invention contemplates the use of a compound(s) as disclosed herein when delivered at a dose of about 0.001 ng/kg. to about 100 ng/kg body weight, from about 0.01 to about 10 ng/kg body weight of about 0.001 μg/kg. to about 100 μg/kg body weight, from about 0.01 to about 10 μg/kg about 0.001 mg/kg to about 100 mg/kg body weight, from about 0.01 to about 10 mg/kg body weight. The compound(s) as disclosed herein can be delivered up to several times per day, as needed. Treatment can be continued, indefinitely to for example normalize mucosal hydration or sodium absorption or reduce excessive mucosal viscosity.

The amount of active ingredient that may be combined with the carrier materials to produce a single dosage form will vary depending upon the host treated and the particular mode of administration.

It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy.

While the compounds of the invention can be administered as the sole active pharmaceutical agent, they can also be used in combination each other and/or with one or more other agents used in the treatment of the symptoms of Covid-19, pulmonary edema, cystic fibrosis, chronic bronchitis, asthma, inflammation and the like.

For example, alleviating pulmonary edema caused by for instance Covid-19, a composition of the present disclosure may be administered that comprises a compound(s) as disclosed herein and a diuretic, such as furosemide (Lasix), to decrease the pressure caused by excess fluid in heart and lungs. Morphine to relieve shortness of breath associated with pulmonary edema and/or blood pressure medications may be administered in compositions comprising the compound(s) disclosed herein.

To alleviate mucosal viscosity, a composition of the present disclosure may be administered that comprises a compound(s) of the disclosure together with an agent useful for the treatment of inflammation-accompanying condition. For instance, such an agent can be mucolytics (e.g., Pulmozyme® and Mucomyst®), purinergic receptor agonists such as uridine triphosphate (UTP), and/or agents for the treatment of any accompanying infection such as tobramycin or aerosolized tobramycin (Tobi™), meropenem, RSV vaccine, IB605, Pa1806, anti-inflammatory agents such as DHA, rHEI, DMP777, IL10 (Tenovil) and/or agents triggering alternate chloride channels such as antibiotics such as Duramycin (Moli901-Molichem Medicines), or omeprazole, and/or purinergic agonists such as nucleotide or dinucleotide analogs, or agents affecting sodium transport such as amiloride, and/or agents affecting pH such as organic acids.

For the treatment of asthma, such agents can be corticosteroids-such as fluticasone propionate (Flovent®, Flovent Rotadisk®), budesonide (Pulmocort Turbuhaler®), flunisolide (Aerobid®), triamcinolone acetonide (Azmacort®), beclomethasone MDI (Beclovent®), antileukotrienes such as Zafirlukast (Accolate®, Zeneca®), Zileuton (Zyflo®), Montelukast or other therapies such as methotrexate, troleandomycin, gold, cyclosporine, 5′-lipoxygenase inhibitors, bronchodilators, or immunotherapeutic agents.

The compounds disclosed herein may also be administered in combination with one or more sodium channel blockers. Sodium channel blockers which may be used in the present invention are typically pyrazine diuretics such as amiloride, as described in U.S. Pat. No. 4,501,729. The term “amiloride” as used herein includes the pharmaceutically acceptable salts thereof, such as (but not limited to) amiloride hydrochloride, as well as the free base of amiloride. The quantity of amiloride included may be an amount sufficient to achieve dissolved concentrations of amiloride on the airway surfaces of the subject of from about 10−⁷ to about 10−³ moles/liter, and more preferably from about 10−⁶ to about 10−⁴ moles/liter.

The methods of the present disclosure may also further comprise the step of removing retained mucus secretions from the lungs of the subject prior to the step of administering the active agent. This facilitates application of the active agent to the respiratory epithelia during the administering step. Such removal of retained mucus secretions can be carried out by any suitable means, including postural drainage, antibiotic administration (e.g., intravenous or inhalation administration of cephalosporin or aminoglycoside antibiotics such as Tobramycin), and/or inhalation administration of DNase. In addition, the compound(s) of the present disclosure may be carried out on patients such as children prior to decline of respiratory function (e.g., patients essentially free of lung blockage due to retained mucus secretions). Such patients can be genetically predisposed to becoming afflicted with lung disease (e.g., cystic fibrosis) as hereinbefore described.

Alternatively, the compositions comprising a compound of the present disclosure can be administered in combination with, prior to, concurrent with or subsequent to the administration of another agent useful for the treatment of any of the accompanying condition of respiratory diseases, as described above.

Compounds of the present disclosure may be tested in vivo to demonstrate efficacy of the compounds in remediating the symptoms of pulmonary edema.

The compounds of the present invention can be used in the form of salts derived from inorganic or organic acids. These salts include but are not limited to the following: acetate, adipate, alginate, citrate, aspartate, benzoate, benzenesulfonate, bisulfate, butyrate, camphorate, camphor-sulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-napthalenesulfonate, oxalate, pamoate, pectinate, persulfate, 3-phenylproionate, picrate, pivalate, propionate, succinate, tartrate, thiocyanate, p-toluenesulfonate and undecanoate.

Examples of acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. Basic addition salts can be prepared in situ during the final isolation and purification of the compounds of formula (IV), or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

The compounds of the invention are useful in vitro for modulating sodium ion absorption in a cell or tissue, and in vivo in human and animal hosts for the regulation of the sodium channel, ENaC. The compounds may be used alone or in compositions together with a pharmaceutically acceptable carrier.

Thus, in one aspect, the present invention provides methods of preventing and/or treating respiratory disease, such as, but not limited to SARS-CoV-2 (Covid-19), cystic fibrosis, bronchiecstasis, chronic bronchitis, and chronic obstructive pulmonary disease (COPD), in a subject in need of such treatment by administering a compound or compositions disclosed herein to the subject in an amount effective to modulate ENaC activity. In another aspect, the present disclosure provides methods of preventing and/or treating respiratory disease such as, but not limited to SARS-CoV-2 (Covid-19), cystic fibrosis, bronchiecstasis, chronic bronchitis, and chronic obstructive pulmonary disease (COPD) in a subject in need of such treatment by administering compound or composition as disclosed herein to the subject in an amount effective to modulate epithelial sodium ion absorption. In another aspect, the present disclosure provides methods of regulating fluid retention by administering compound or composition as disclosed herein to the subject in an amount effective to modulate epithelial sodium ion absorption. In yet other aspects, the present disclosure provides methods of use of the active compounds as disclosed herein for the manufacture of a medicament for the prophylactic or therapeutic treatment of respiratory disease, such as, but not limited to SARS-CoV-2 (Covid-19), cystic fibrosis, bronchiecstasis, chronic bronchitis, and chronic obstructive pulmonary disease (COPD) in a subject in-need of such treatment.

The embodiments described herein are intended to be merely exemplary, and those skilled in the art will recognize, or be able to ascertain using no more than routine experimentation, numerous equivalents to the specific procedures described herein. All such equivalents are considered to be within the scope of the present invention and are covered by the following embodiments.

All references (including patent applications, patents, and publications) cited herein are incorporated herein by reference in their entirety and for all purposes to the same extent as if each individual publication or patent or patent application was specifically and individually indicated to be incorporated by reference in its entirety for all purposes.

### EXAMPLES

In order that this invention be more fully understood, the following examples are set forth. These examples are only for the purpose of illustration and are not to be construed as limiting the scope of the invention in any way.

### Example 1

### Identifying and Testing of Compounds for Preventing and/or Treating SARS-CoV-2 (COVID-19)

### Primary and Secondary Cell-Based Testing:

Compound(s) disclosed herein may be tested in cell-based assays using cells expressing one or more ENaC subunits, including primary cells, cells endogenously expressing the one or more subunits and/or cells transiently or stably transfected to express the one or more subunits. The cell based assays may be used to provide proteolyzed forms of the one or more ENaC subunits. This may be achieved by introducing in the cell based assay a step wherein the cells are exposed to varying concentrations of one or more proteases for varying periods of time from less than 1 minutes, 1 minute to 60 minutes, 1 hour to 24 hours and from 4 degrees C. to 42 degrees C. or higher. ENaC expressed in the cells of the cell-based assays may be proteolyzed using one or more purified proteases including proteases expressed or secreted into the lung, respiratory tract, GI tract, stomach, musculature or oral cavity as well as proteases secreted by cells, including cells of the immune system and during inflammatory responses. ENaC expressed in the cells of the cell-based may also be proteolyzed using protease contained in, derived, obtained or purified from fluid, excretions or material obtained from humans or animal organs and cells, including lungs, the GI tract, stomach, musculature, heart, oral cavity, immune system and cells of the immune system. ENaC expressed in the cells of the cell-based may also be proteolyzed by proteases expressed by the cells or provided in the cell culture medium. Cell-based assays may also be used to test compounds for activity against one or more proteolyzed variants of ENaC by providing cells for use in the cell-based assay where the cells are created to express fragments of the one or more ENaC subunits corresponding to the proteolyzed forms of ENaC that result following proteolysis by one or more proteases.

The ENaC cell-based may be used to test compounds for activity against one, a subset or all forms of ENaC, including any one or more subunits, heteromultimeric combinations comprising different stoichiometries of the subunits, post-translationally modified or proteolyzed variants, including non-proteolyzed variants of the one or more ENaC subunits. In all of the above, the one or more subunits of ENaC may correspond to rodent, mouse, hamster, rat, rabbit, guinea pig, ferret, dog, cat, pig, sheep, goat, horse, cow, primate, monkey, chimpanzees and/or human ENaC sequences, or any combination of these.

Laboratory cells, cell cultures or primary cells may be used to create the ENaC cell-based assays. The cells used to create the cell based assays may be of mammalian, reptile, avian or marsupial origin, including cells originating, derived or obtained from rodent, mouse, hamster, rat, rabbit, guinea pig, ferret, dog, cat, pig, sheep, goat, horse, cow, primate, monkey, chimpanzees and/or human origin.

The cells may be tested using high throughput screening (HTS) assays, plate-readers, Using chamber systems and other secondary cell-based assay methodologies. Cell-based assays may be created using membrane potential dyes and/or other fluorescent reporter dyes and adapted to HTS format, including 96-well, 384-well, 1536-well format using fluorescent plate readers.

The compounds disclosed herein that are found to modulate ENaC may be prioritized for further testing. The cell-based assays may be used to identify positive and negative modulators including blockers, inhibitors, activators, potentiators, agonists, partial agonists, modulators, allosteric modulators and others.

### Animal Models

Compounds may be evaluated for efficacy in preventing and/or treating respiratory disease, such as, but not limited to SARS-CoV-2 (Covid-19), pulmonary edema, cystic fibrosis, SARS (severe acute respiratory syndrome), MERS (middle east respiratory syndrome), ARDS (acute respiratory distress syndrome), pneumonia, infection, pulmonary infection, viral infection, bronchiectasis, chronic bronchitis, and chronic obstructive pulmonary disease (COPD), high altitude pulmonary edema (HAPE) using suitable animal models, such as, but not limited to rodents, mice, hamsters, rats, rabbits, guinea pigs, ferrets, dogs, cats, pigs, sheep, goats, horses, cows, primates, monkeys, and chimpanzees. The choice of the model will depend on which model best reproduces one or more symptoms associated with a respiratory disease, such as, but not limited to SARS-CoV-2 (Covid-19), cystic fibrosis, SARS (severe acute respiratory syndrome), MERS (middle east respiratory syndrome), ARDS (acute respiratory distress syndrome), pneumonia, infection, pulmonary infection, viral infection, bronchiectasis, chronic bronchitis, and chronic obstructive pulmonary disease (COPD), or high altitude pulmonary edema (HAPE).

### Viral Models

Compounds may be evaluated for possible efficacy against SAR-CoV-2 infection using in vitro or cell-based and animal models. Compounds may be evaluated for possible intervention or perturbation of one or more steps or life-cycle stages of coronavirus or SARS-CoV-2 or related virus using in vitro or cell-based and animal models. Coronaviral proteins have previously been reported to interact with ENaC and/or modulate ENaC activity. Therefore, compounds may be evaluated for both their mechanism of activity against SARS-CoV-2 as well as for activity in any of the cell-based and animal models above or using in vitro assays. Compounds may be evaluated for possible direct or indirection interaction with one or more proteins of coronavirus or SARS-CoV-2 or related virus using in vitro or cell-based and animal models. Similarly, compounds may be tested for their efficacy against other viral infections using in vitro or cell-based and animal models.

### Compound Optimization

Once a lead compound has been identified, the active part of the molecule that interacts with the biological target of the molecule can be identified. Analogs of the lead compound can be created, using methods known to those of skill in the art, by changing sites of the molecule that are not part of the active core to decrease or increase the effectiveness of the compound(s).

Compounds may also be optimized for activity against one or more of the cell-based or animal models above by creating and testing analogs. Compounds may also be optimize for stability, solubility, delivery, safety, efficacy, cost, synthesis or scale-up.

### Safety and Tox Studies

Once a compound(s) has been identified for further drug development, non-clinical studies are conducted using different protocols including animal studies, which will follow mostly the Good Laboratory Practice (GLP) regulations. During the early pre-clinical development process, the drug candidate will pass through several steps, such as determination of drug availability (studies on pharmacokinetics), absorption, distribution, metabolism and elimination (ADME) and preliminary studies that aim to investigate the candidate safety including genotoxicity, mutagenicity, safety pharmacology and general toxicology. These preliminary studies generally will not need to comply with GLP regulations. These studies aim at investigating the drug safety to obtain the first information about its tolerability in different systems that are relevant for further decisions. Other studies will be subsequently performed following the GLP standards to assure safe exposure to humans, such as repeated dose toxicity, genotoxicity and safety pharmacology. These studies will be conducted before the Investigational New Drug (IND) application. The package of non-clinical studies will cover all information needed for the safe transposition of drugs from animals to humans, generally based on the non-observed adverse effect level obtained from general toxicity studies. After IND approval, other GLP experiments for the evaluation of chronic toxicity, reproductive and developmental toxicity, carcinogenicity and genotoxicity, will be carried out during the clinical phase of development.

What is claimed is:

1. A composition comprising a compound of Formula (IV) 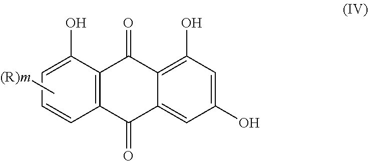
or a comestibly or biologically acceptable salt, or an enantiomer, diastereomer, or solvate thereof, or a combination of any of the foregoing compounds; wherein as valence and stability permit:
R is each independently hydrogen, halogen, C1-C6 alkyl, C1-C6 alkoxy, or hydroxyl;
m is 1, 2, or 3; or
and at least one pharmaceutically acceptable carrier or diluent.

2. The composition of claim 1 wherein the compound of Formula (IV) is a compound wherein R of the compound of Formula (IV) is —CH3 and m is 1.

3. The composition of claim 1 wherein the compound of Formula (IV) is 1,3,8-trihydroxy-6-methylanthraquinone (Compound 1), having the following structure:

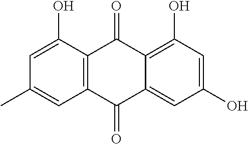 [SMILES: Cc1cc(O)c2c(c1)C(=O)c1cc(O)cc(O)c1C2=O]

4. The composition of claim 1 wherein the at least one compound is Emodin.

5. The composition of claim of any one of claim 1-4 wherein the composition is in an aqueous solution.

6. The composition of any one of claims 1-5 further comprising one or more other active agents selected from the group of antibiotics, vaccines, decongestants (nasal or bronchial), rhDNase, non-steroidal antiinflammatory agents (NSAIDs), steroids, antiviral agents, elastase inhibitors, gene therapy agents, chloride channel activators and bronchodilators.

7. The composition of any one of claims 1-6 for use in a method of preventing or treating respiratory disease.

8. The composition of claim 7 wherein the respiratory disease is characterized by one or more of the symptoms selected from fluid dysregulation, pulmonary edema, lack of fluid clearance, excessive fluid build-up, poor mucociliary clearance, mucostasis, breathing difficulties, cough, chest pain, and fatigue.

9. The composition of claim 7 or 8 wherein the respiratory disease is selected from SARS-CoV-2 (Covid-19), pulmonary edema, cystic fibrosis, SARS (severe acute respiratory syndrome), MERS (middle east respiratory syndrome), ARDS (acute respiratory distress syndrome), pneumonia, infection, pulmonary infection, viral infection, bronchiectasis, chronic bronchitis, and chronic obstructive pulmonary disease (COPD), high altitude pulmonary edema (HAPE).

10. The composition of claim 9 wherein the respiratory disease is SARS-CoV-2 (Covid-19).

11. The composition of any one of claims 1-10 wherein the composition is formulated for administration to the respiratory system by the pulmonary route.

12. The composition of claim 11 wherein the composition is formulated for administration by any one of endotracheal intubation, intratracheal installation, intratracheal delivery of lipososmes, insufflation, nebulization, dry powder inhalation and aerosol inhalation.

13. The composition of any one of claims 1-1o wherein the composition is formulated for administration by diffusion in the air.

14. The composition of any one of claims 1-10 wherein the composition is formulated for administration by infusion into cloth selected from tissues, handkerchiefs, napkins, face masks, mufflers, scarfs, wraps, bandanas, ties, shawls, hijabs, and veils.

15. The composition of any one of claims 1-10 wherein the composition if formulated for administration by injection, oral or rectal route.

16. The composition of any one of claims 1-12 wherein the composition is administered by inhalation.

17. The composition of claim 16 wherein the composition is formulated for dry powder inhalation or aerosol inhalation, together with a propellant selected from hydrofluroalkanes, chlorofluorocarbons, propane and nitrogen, or mixtures thereof.

18. An inhalation device loaded with the pharmaceutical composition of any one of claim 1-12 or 16-17.

19. The inhalation device of claim 18, wherein the device is a dry powder inhaler, metered dose inhaler, jet nebulizer or ultrasonic nebulizer.

20. A method of treating a respiratory disease, the method comprising administering to a subject in need of such treatment a therapeutically effective amount of a compound of Formula (IV), or Compound 1; or a comestibly or biologically acceptable salt, or an enantiomer or diastereomer thereof, or a combination of any of the foregoing compounds.

21. The method of claim 20 wherein the compound is Emodin, or a pharmaceutically acceptable salt, or a stereoisomeric or tautomeric form thereof; or a combination of any of the foregoing compounds.

22. The method of any one of claims 20-21, wherein said compound is an ENaC agonist.

23. The method of any one of claims 20-22, wherein the respiratory disease is characterized by one or more of the symptoms selected form poor mucociliary clearance, mucostasis, breathing difficulties, cough, chest pain, and fatigue.

24. The method of any one of claims 20-23, wherein the respiratory disease is selected from SARS-CoV-2 (Covid-19), cystic fibrosis, bronchiecstasis, chronic bronchitis, and chronic obstructive pulmonary disease (COPD).

25. The method of claim 24 wherein the respiratory disease is SARS-CoV-2 (Covid-19).

26. The method of any one of claims 20-25 wherein the administration is to the respiratory system by the pulmonary route.

27. The method of claim 26 wherein the administration is by one or more treatments selected from endotracheal intubation, intratracheal installation, intratracheal deliver of lipososmes, insufflation, nebulization, dry powder inhalation and aerosol inhalation.

28. The method of claim 26 wherein the administration is by dry powder inhalation or aerosol inhalation, together with a propellant selected from hydrofluroalkanes, chlorofluorocarbons, propane, nitrogen, or a mixture thereof.

29. An ENaC agonist for use in a method of treating a respiratory disease.

30. The ENaC agonist of claim 29 wherein said agonist is selected from modulators, including allosteric modulators, potentiators, enhancers, activators, and blockers, or any combination of the foregoing.

31. The ENaC agonist of claim 29 or 30 wherein the agonist is an ENaC enhancer.

32. The ENaC agonist of claim 31 wherein the agonist is selected from a compound of Formula (IV), Emdodin, and Compound 1; or a comestibly or biologically acceptable salt, or an enantiomer or diastereomer thereof, or a combination of any of the foregoing compounds.

33. The ENaC agonist of claim 32 wherein the agonist is from Emodin.

34. The ENaC agonist of any one of claims 29-33 wherein the agonist further comprises one or more pharmaceutically acceptable carriers or diluents.

35. The ENaC agonist of any one of claims 29-34 wherein the agonist is in an aqueous solution.

36. The ENaC agonist of any one of claims 29-25 wherein the agonist also comprises one or more other active agents selected from antibiotics, vaccines, bronchial decongestants, rhDNase, non-steroidal anti-inflammatory agents (NSAIDs), steroids, antiviral agents, elastase inhibitors, gene therapy agents, chloride channel activators and bronchodilators.

37. The method of any one of claims 20-28, wherein the therapeutically effective amount is effective to alleviate at least one symptom associated with said respiratory disease in a subject, wherein a compound according to the present disclosure, or a pharmaceutically acceptable salt, or a tautomeric form thereof, shows a reduction in at least one symptom associated with said inflammatory condition at a dose between 0.01 ng/kg and 10,000 ng/kg, at a dose between 0.1 ng/kg and 1,000 ng/kg, at a dose between 0.5 ng/kg and 100 ng/kg, or at a dose between 1 ng/kg to 50 ng/kg between 0.01 μg/kg and 10,000 μg/kg, at a dose between 0.1 μg/kg and 1,000 μg/kg, at a dose between 0.5 μg/kg and 100 μg /kg, or at a dose between 1 μg/kg to 50 μg/kg, or at a dose between 0.01 mg/kg and 10,000 mg/kg, at a dose between 0.1 mg/kg and 1,000 mg/kg, at a dose between 0.5 mg/kg and 100 mg/kg, or at a dose between 1 mg/kg to 50 mg/kg.

38. Use of a compound selected from:
a compound of Formula (IV), Emodin and Compound 1; or a comestibly or biologically acceptable salt, or an enantiomer or diastereomer thereof, or a combination of any of the foregoing compounds for the manufacture of a medicament for the treatment of respiratory disease.

39. The use of a compound of claim 28 wherein the compound is Emodin.

40. An article of manufacture comprising packaging material and a pharmaceutical agent contained within said packaging material, wherein said packaging material comprises a label which indicates said pharmaceutical may be administered, for a sufficient term at an effective dose, for treating respiratory conditions together with a pharmaceutically acceptable carrier, wherein the pharmaceutical agent comprises a compound, or a compound selected from: a compound of Formula (IV), and Compound 1; or a pharmaceutically acceptable salt, or a tautomeric form thereof.

41. The article of manufacture of claim 40 wherein the compound is Emodin.
